(19) United States
(12) Patent Application Publication
(10) Pub. No.: US 2012/0136329 A1
(43) Pub. Date: May 31, 2012

(54) ABSORBENT ARTICLE WITH AN ACQUISITION DISTRIBUTION LAYER WITH CHANNELS

(75) Inventor: Joshua CARNEY, Philadelphia, PA (US)

(73) Assignee: SCA HYGIENE PRODUCTS AB, Goteborg (SE)

(21) Appl. No.: 12/956,482

(22) Filed: Nov. 30, 2010

(51) Int. Cl. A61F 13/512

(52) U.S. Cl. 604/383

(57) ABSTRACT

An absorbent article, such as a sanitary napkin, includes a top sheet and a back sheet with an absorbent core sandwiched therebetween. To decrease transverse leakage, a channeled acquisition distribution layer is placed between the absorbent core and the top sheet. The acquisition distribution layer has apertures and channels open toward the top sheet extending in the longitudinal or machine direction of the article. The channels have a lower permeability through the layer than the remaining portions of the acquisition distribution layer by containing less apertures or by melting the channels so that material fills in substantially all of the apertures in the channels. In further embodiments, the channels may be impermeable and extend substantially the full length of the acquisition distribution layer.

### FIELD OF THE INVENTION

The present invention pertains to an absorbent article having a channeled transfer layer. More particular, the present invention pertains to an absorbent article having channels in the transfer layer that have a lower permeability than the portions of the transfer layer outside the channels.

### BACKGROUND OF THE INVENTION

In the discussion of the background that follows, reference is made to certain structures and/or methods. However, the following references should not be construed as an admission that these structures and/or methods constitute prior art. Applicant expressly reserves the right to demonstrate that such structures and/or methods do not qualify as prior art.

A variety of absorbent articles that are adapted to absorb body fluids are well known. Examples of absorbent articles include diapers, feminine hygiene products, and incontinence guards. These products typically include a top sheet facing the body of the user, a back sheet facing the garment of a user, and an absorbent layer sandwiched between the inner, top sheet and outer, back sheet.

One problem associated with known absorbent articles is waste product leakage, which may contaminate bedding and clothing articles, such as pants and shirts. The amount of leakage experienced by a wearer can be reduced by increasing the rate that liquid enters the absorbent core. Therefore, an absorbent article wherein liquid rapidly penetrates the topsheet and is contained in the absorbent core will experience less leakage than an absorbent article wherein liquid is able to run across the topsheet before penetrating into the absorbent core. Consequently, run-off reduction reduces the amount of leakage associated with an absorbent article.

Another problem associated with absorbent articles is dryness of the skin contacting surface of the article. Generally, the drier the skin contacting surface, the more comfortable the absorbent article. Therefore, more separation between the absorbent core and the skin contacting surface is typically desired.

An additional problem with typical absorbent articles is transverse liquid leakage. Liquid discharged form the body may pass through the topsheet mainly in a central portion of the absorbent article for subsequent absorption and retention by the absorbent layer. Upon subsequent discharge, the central portion becomes saturated, and fluid begins to flow away from the saturated central portion. If the fluid flows in a transverse direction as opposed to the desired longitudinal direction, the fluid may leak out of the absorbent article rather than flowing to the areas of unsaturated core material. This may cause premature leakage, prior to filling the absorbent core.

U.S. Patent Application Publication No. 2002/0062113 discloses an absorbent article with an acquisition distribution layer used to achieve a reduced surface run-off. The acquisition distribution layer is made of a three dimensional apertured polymeric film. The underside of the acquisition layer defines a high void volume space that provides space for unabsorbed fluid to flow over the top plane of saturated core regions and flow to new, unsaturated regions of the core material without contacting the topsheet. This high void volume space under the acquisition layer may provide further separation between the flowing fluid and the wearer, but it fails to prevent transverse liquid leakage.

Therefore, there is still a need for a quicker absorbing article having higher levels of dryness that also prevents transverse liquid leakage.

### SUMMARY

It has been discovered that an acquisition distribution layer as described below can achieve the above-mentioned combination of reduced surface run-off and prevention of transverse liquid leakage, which can be caused by one portion of the absorbent core becoming saturated before the majority of other portions of the absorbent core. The acquisition distribution layer dispenses the fluid over a greater area of the absorbent core than in other known absorbent articles. The acquisition distribution layer includes channels with very low or no permeability. Fluid in the channels travel down the channels and away from the insult area. Thus, the inlet time is decreased, and more of the core pre-insult is used.

According to a first aspect of the present invention, an absorbent product comprises a backsheet, a topsheet, an absorbent core between the topsheet and backsheet, and an acquisition distribution layer including a first plane between the topsheet and absorbent core. The acquisition distribution layer has apertures and channels. The channels are open toward the top sheet and extend in the longitudinal direction of the acquisition distribution layer. The bottom of the channels are situated in a lower second plane, that is closer to the core than the first plane. The channels have a lower permeability through the layer than other portions of the acquisition distribution layer.

According to a second aspect of the present invention, an absorbent product comprises a backsheet, a topsheet, an absorbent core between the topsheet and backsheet, and an acquisition distribution layer between the topsheet and absorbent core. The acquisition distribution layer has apertures and channels. The channels are open toward the top sheet and extend in the longitudinal direction of the acquisition distribution layer. The bottom of the channels are situated in a lower second plane, that is closer to the core than the first plane. The channels are impermeable and contain no apertures.

Yet further features that apply to the first and second aspect described above are described below.

Yet another feature of the present invention pertains to an absorbent product wherein the acquisition distribution layer includes three or more channels extending in the longitudinal direction.

Yet another feature of the present invention pertains to an absorbent product wherein the channels are straight, curved, jagged, or S-curved.

Yet another feature of the present invention pertains to an absorbent product wherein the channels have a length from about 60% to about 100% of a total length of the acquisition distribution layer, and more particularly from about 80% to about 100%. In yet more particular, the channels extend the full length of the acquisition distribution layer.

Yet another feature of the present invention pertains to an absorbent product wherein the acquisition distribution layer has a length from about 25% to about 100% of the length of the absorbent core, and more particularly from about 50% to about 80%.

Yet another feature of the present invention pertains to an absorbent product wherein the channels have a width of from about 1 mm to about 3 mm.

Yet another feature of the present invention pertains to an absorbent product wherein the channels have a depth of from 0.5 mm to about 1.5 mm.

Yet another feature of the present invention pertains to an absorbent product wherein the apertures situated in the first plane have a hole size from about 0.25 mm to about 1.5 mm.

Yet another feature of the present invention pertains to an absorbent product wherein the apertures are ovals, squares, or circles, or combinations thereof.

Yet another feature of the present invention pertains to an absorbent product wherein the acquisition distribution layer has from 5 to 150 apertures per square centimeter, and more particularly from 5 to 100 apertures per square centimeter.

Yet another feature of the present invention pertains to an absorbent product wherein the apertures are arranged in a grid pattern or in an offset pattern.

Yet another feature of the present invention pertains to an absorbent product wherein the apertures are conical with a larger surface area on the surface facing the topsheet.

Yet another feature of the present invention pertains to an absorbent product wherein the apertures are conical with a larger surface area on the surface facing the absorbent core.

Yet another feature of the present invention pertains to an absorbent product wherein the apertures have a depth from about 0.2 mm to about 1 mm.

Yet another feature of the present invention pertains to an absorbent product wherein the channels contain a smaller surface area of apertures than the other portions of the acquisition distribution layer.

Yet another feature of the present invention pertains to an absorbent product wherein the apertures within the channels have a hole size from about 0.05 mm to about 0.5 mm.

Yet another feature of the present invention pertains to an absorbent product wherein the channels have from 1 to 20 apertures per square centimeter.

Yet another feature of the present invention pertains to an absorbent product wherein the apertures within the channels are separated by a hole spacing from about 1 mm to about 50 mm.

Yet another feature of the present invention pertains to an absorbent product wherein the apertures within the channels are arranged in a grid pattern or in an offset pattern.

Yet another feature of the present invention pertains to an absorbent product wherein the channels contain no apertures.

Yet another feature of the present invention pertains to an absorbent product wherein the acquisition distribution layer is formed from a three-dimensional apertured polymeric film.

Yet another feature of the present invention pertains to an absorbent product wherein the three-dimensional apertured polymeric film has a weight from about 10 to about 50 grams per square meter.

Yet another feature of the present invention pertains to an absorbent product wherein the three-dimensional apertured polymeric film is formed from polypropylene or polyethylene.

Yet another feature of the present invention pertains to an absorbent product wherein the acquisition distribution layer is formed of a non-woven material.

Yet another feature of the present invention pertains to an absorbent product wherein the non-woven material is spunbond-meltblown-spunbond (SMS) non-woven.

Yet another feature of the present invention pertains to an absorbent product wherein the non-woven material has a weight from about 15 to about 50 grams per square meter.

Yet another feature of the present invention pertains to an absorbent product wherein the non-woven material has a pre-apertured hydrohead value from about 15 to about 23 mbar.

Yet another feature of the present invention pertains to an absorbent product wherein the channels are formed by melting the non-woven material to lower the permeability of the non-woven material in the channels.

Yet another feature of the present invention pertains to an absorbent product wherein the channels are substantially impermeable.

Yet another feature of the present invention pertains to an absorbent product wherein the absorbent article is an incontinence pad or a sanitary napkin.

It is to be understood that both the foregoing general description and the following detailed description are exemplary and explanatory and are intended to provide further explanation of the invention as claimed.

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements and:

FIG. 1 illustrates a top view of an exemplary absorbent article according to the features of the invention.

FIG. 2 illustrates an exploded view of the absorbent article of FIG. 1.

FIG. 3 illustrates a perspective closer view of portions of an acquisition distribution layer and absorbent core present in the absorbent article of FIG. 1.

FIG. 4 is a cross-sectional view cut along line IV-IV of the acquisition distribution layer and absorbent core of FIG. 3.

FIG. 5 is a top view of the acquisition distribution layer of FIG. 3.

FIG. 6 illustrates a perspective view of an acquisition distribution and absorbent core according to a second embodiment of the invention.

FIG. 7 is a cross-sectional view cut along line VII-VII of the acquisition distribution layer and absorbent core of FIG. 6.

FIG. 8 illustrates a cross-sectional view of an acquisition distribution layer and absorbent core according to a third embodiment of the invention.

FIG. 9 is a close up view of a longitudinal end portion of an acquisition distribution layer according to a fourth embodiment of the invention.

FIG. 10 illustrates anticipated dispersion of excess fluids contacting an acquisition distribution layer in accordance with a further embodiment of the present invention.

FIG. 11 illustrates a top view of an acquisition distribution layer according to a fifth embodiment of the invention.

FIG. 12 illustrates a top view of an acquisition distribution layer according to a sixth embodiment of the invention.

FIG. 13 illustrates a top view of an acquisition distribution layer according to a seventh embodiment of the invention.

FIG. 14 illustrates a perspective view of an acquisition distribution layer and absorbent core according to an eight embodiment of the invention

### DETAILED DESCRIPTION OF THE PREFERRED EMBODIMENTS

The present invention pertains to an absorbent article. An absorbent article is defined as an article or garment used for the absorption of body fluids, including but not limited to, infant diapers and training pants, adult incontinence products, feminine hygiene products, gender specific absorbent products, and pet training absorbent articles. While the Figures do not illustrate every type of absorbent article, it should be understood that the present invention pertains to all types of absorbent articles.

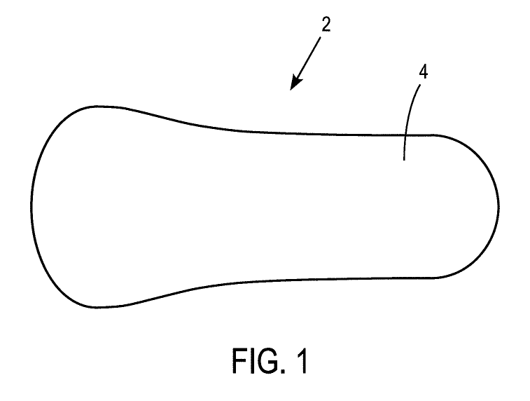

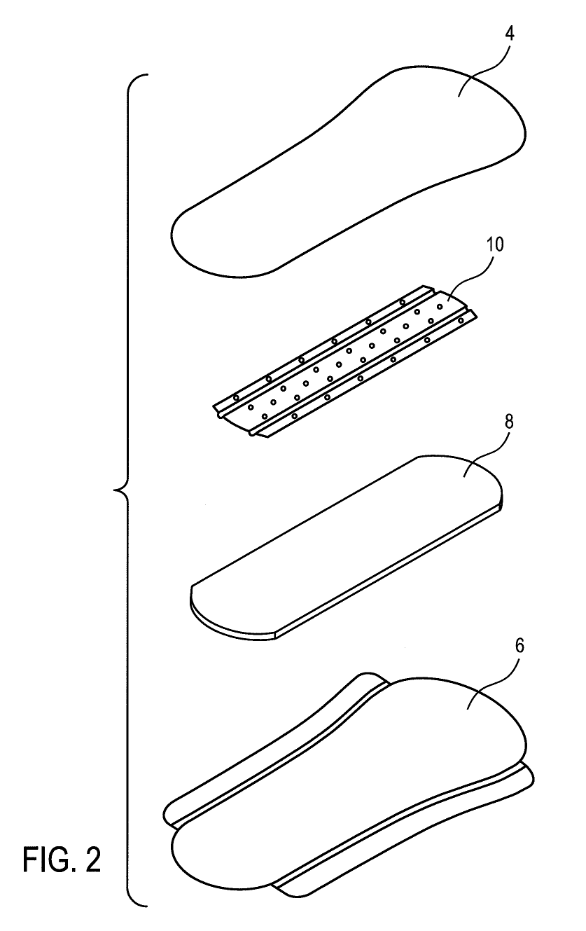

With reference to FIGS. 1 and 2, an absorbent article 2 includes a back sheet 6 and a top sheet 4 which sandwich an absorbent core 8 therebetween. Additionally, the absorbent article 2 includes an acquisition distribution layer 10 between the top sheet 4 and the absorbent core 8. The acquisition distribution layer 10 is specially designed to acquire fluids that pass through the top sheet 4 and distribute the fluids to the absorbent core 8. The topsheet 4 can be joined together with the backsheet 6 and the absorbent core 8 and acquisition distribution layer 10, for example by means of adhesive, ultrasonic joining or by means of some form of thermal bonding.

Preferably, the absorbent core 8 is appropriately manufactured from a suitable fiber material in the form of natural or synthetic fibers having absorbent properties, or a mixture of natural fibers and synthetic fibers or other absorbent materials of a previously disclosed kind that are suitable for use in sanitary towels, incontinence pads and panty liners, for example. The absorbent core 8 can also contain a predetermined proportion of superabsorbent polymers (SAP). SAP materials are in the form of particles, fibers, flakes or the like, and have the capacity to absorb and to chemically bind liquid equivalent to several times their own weight while forming an aqueous gel. This provides a very high water-absorbent capacity in the finished product.

The liquid-permeable topsheet 4 includes one or more layers of one or more of the following materials: a fibrous material, for example a soft nonwoven material, plastic film, mesh, open-celled foam, material laminate, etc. The topsheet 4 may include a perforated plastic film, for example, a thermoplastic plastic material such as polyethylene or polypropylene, or a mesh-like layer of synthetic or textile material. Synthetic mono-, bi-, or multi-component fibers, made of polymers such as polyethylene, polypropylene, polyester, nylon or the like, are preferably used as a nonwoven material. Mixtures of different types of fibers can also be used for the aforementioned nonwoven material.

The backsheet 6 is preferably liquid-impermeable (or at least possesses high resistance to penetration by liquid) and is thus so arranged as to prevent any leakage of excreted fluid from the product. The backsheet 6, on the other hand, may also be vapor-permeable. The backsheet 6 may be manufactured from a liquid-impermeable material which includes a thin and liquid-proof plastic film. For example, plastic films of polyethylene, polypropylene or polyester can be used for this purpose. Alternatively, a laminate of nonwoven and plastic film or other suitable layers of material can be used as a liquid-proof backsheet 6. In a previously disclosed manner, the clothing side of the backsheet 6 can be provided with beads of adhesive or some other previously disclosed attachment means, which can then be utilized for the application of the product to an item of clothing. The product can also be provided with wings, that is to say folding flaps which are arranged along the sides of the product and can be utilized in conjunction with the application of the product.

With reference to FIG. 2, the absorbent article 2 of the present invention is illustrated in an exploded view. In particular, an acquisition distribution layer 10 may be disposed between the topsheet 4 and the absorbent core 8. The acquisition distribution layer 10 may be in the form of a three-dimensional layer having apertures 14. The acquisition distribution layer 10 further includes channels 12 open toward the topsheet 4 and that extend in the longitudinal direction. The channels 12 have lower permeability than other portions of the acquisition distribution layer 10. In certain embodiments, the channels 12 are impermeable to fluids and contain no apertures.

The acquisition distribution layer 10 may have an appropriately specified thickness and resilience. The acquisition distribution layer 10 may include two dimensional or three dimensional apertures. In certain embodiments, the acquisition distribution layer 10 includes three dimensional apertures. Some exemplary three dimensional apertured films that could be used include, for example, films disclosed in EP 0 057 484 A2 or WO 97/03818.

The acquisition distribution layer 10 may be in the form of a polymeric film or a non-woven material. In certain embodiments, the polymeric films forming the acquisition distribution layer have a weight from about 10 to about 50 grams per square meter. In more certain embodiments, the weight is from about 20 to about 40 grams per square meter. The acquisition distribution layer may be formed from polypropylene or polyethylene. Particular non-woven materials used for the acquisition distribution layer include spunbond-meltblown-spunbond (SMS) non-woven. Non-woven material used may have a weight from about 15 to about 50 grams per square meter.

Further, particular non-woven materials have a pre-apertured hydrohead value from about 15 to about 23 mbar. Hydrohead values can be determined using a Textest FX30000 Hydrostatic Head Tester to test a 5 inch by 5 inch sample. Specifically, the sample is clamped in the tester, which steadily increases test pressure until three leaks are observed, at which point the hydrohead value is determined.

The difference in permeability between the channels 12 and the remaining portions of the acquisition distribution layer 10 is formed by less aperture surface area in the channels or by channels having no apertures. Further, with regard to permeable materials forming the acquisition distribution layer 10, especially with regard to non-woven material, the difference in permeability may be created by melting the permeable material in the channels.

The absorbent article 2 may be made from numerous other layers, as is known in the art. However, the specific layers used may vary, depending on design preference and application. For example, other layers not shown may be used, such as an additional back sheet, or any other intervening layer, such as SMS (Spunbond MeltBlown Spunbond), SSS (three layers of Spunbond), Carded NW, Hydroentangled, and the like. Likewise, the particular number and order of layers is optional, depending on application and design preference.

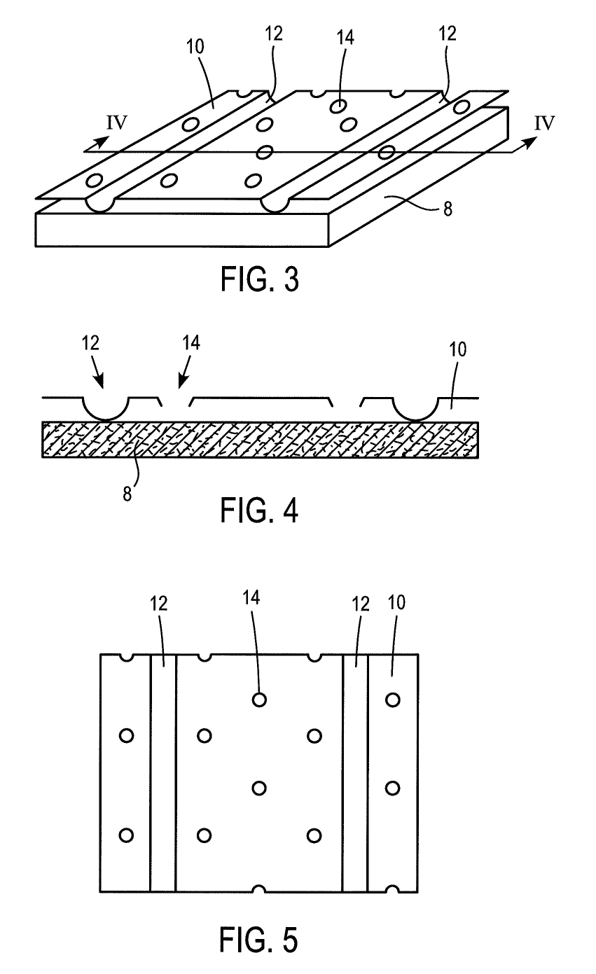

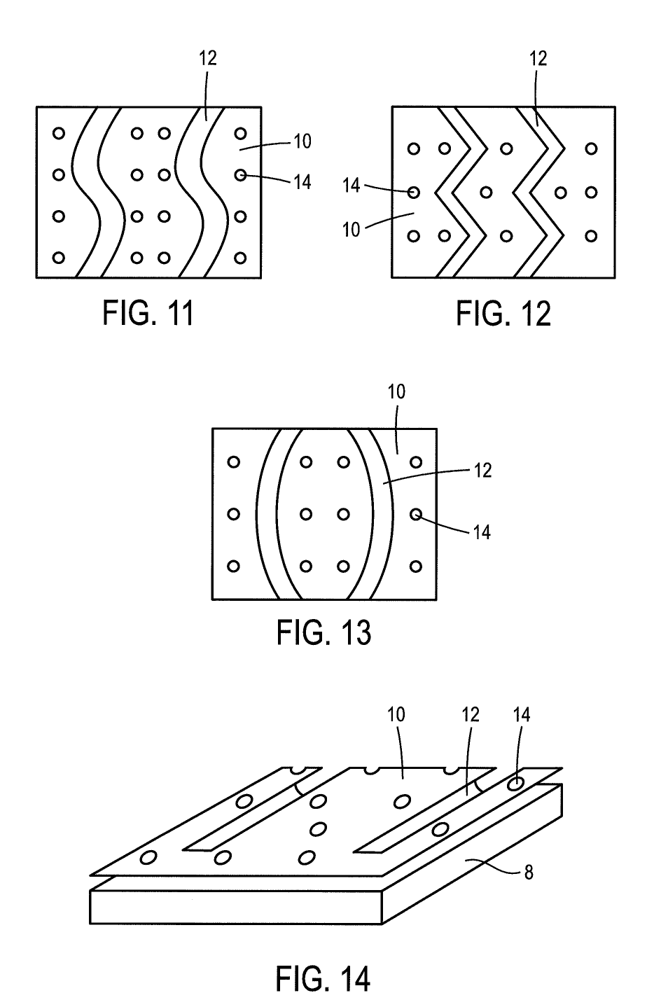

With reference to FIGS. 3-14, various embodiments of the acquisition distribution layer 10 of the present invention will be described in more detail. Generally, the acquisition distribution layer 10 includes longitudinally extending channels. In particular, the acquisition distribution layer includes at least one channel. More particularly, the acquisition distribution layer includes two or more, or even more particularly, three or more channels. Further, in addition to straight longitudinal channels, the channels may also be curved, jagged, or S-curved as illustrated, for example, in FIGS. 11-13.

The channels help distribute fluid to portions of the absorbent core away from the center where the majority of the insult is typically concentrated. To direct the fluids away from the center, the channels may have a length from about 60% to about 100% of a total length of the acquisition distribution layer. In particular, the channels have a length from about 80% to about 100% of the length of the acquisition distribution layer. Yet more particularly, the channels extend the full length of the acquisition distribution layer so that the fluid in the channels run off the longitudinal ends of the acquisition distribution layer. FIGS. 3, 5, 6, and 10-13 illustrate examples of channels that extend the full length of the acquisition distribution layers. Further, FIG. 14 illustrates an embodiment of the acquisition distribution layer i in which channels do not extend the full length of the acquisition distribution layer.

The length of the channels in relation to the acquisition distribution layer is also at least partially dependent on the length of the acquisition distribution layer relative to the absorbent core. In certain embodiments, the acquisition distribution layer has a length from about 25% to about 100% of the length of the absorbent core. In more certain embodiments, the length of the acquisition distribution layer is from 50% to 80% of the length of the absorbent core. In particular embodiments, the channels have a width from about 1 mm to about 3 mm and a depth of from 0.5 mm to about 1.5 mm. In certain embodiments when the acquisition distribution layer is made of a layer with three dimensional apertures, the depth of the channels may be larger than the depth of the apertures and may have a width larger than the apertures in the remaining portions of the acquisition distribution layer. For example, apertures may have a depth from about 0.2 mm to about 1 mm.

The non-channel portions of the acquisition distribution layer have apertures to quickly distribute the fluids into the absorbent core. The apertures may have a hole size from about 0.25 mm to about 1.5 mm. The hole size of the apertures are measured along the largest straight dimension of the aperture. The apertures may have any shape, including ovals, squares, or circles. Further, the same acquisition distribution layer may have a distribution of apertures with different shapes and hole sizes or a distribution of uniform apertures. In many embodiments, uniform apertures are preferred so as to increase the uniformity of fluid distribution to the absorbent core. In certain embodiments, the acquisition distribution layer has from 5 to 150 apertures per square centimeter, or more particularly, 5 to 100 apertures per square centimeter.

With reference to FIGS. 3-5, a first embodiment of the absorbent article and in particular the acquisition distribution layer 10 of the first embodiment of the absorbent article is illustrated. The channels 12 have no apertures, while the non-channeled portions of the acquisition distribution layer 10 include apertures 14 distributed in an offset pattern. Further, the apertures are conical with a larger surface area on the surface facing the top sheet to increase the speed at which the fluids enter the absorbent core.

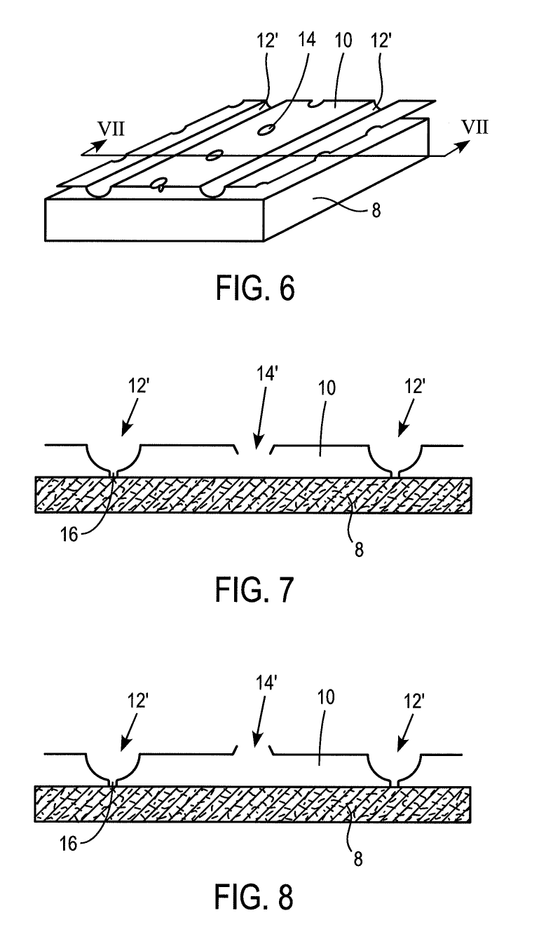

With reference to FIGS. 6-7, a second embodiment of the absorbent article, specifically the acquisition distribution layer 10 of the second embodiment of the absorbent article is illustrated. In the second embodiment, the acquisition distribution layer 10 includes apertures 14 distributed in a grid pattern. Further, the channels 12′ differ from the channels in the first embodiment in that small apertures 16 are formed in the channels themselves. Any apertures 16 formed in the channels are of smaller size and/or distributed with less frequency than apertures 14 formed in the non-channel portions of the acquisition distribution layer 10.

Apertures in the channel portions of the acquisition distribution layers may have a hole size, measured along the largest straight dimension of the aperture, from about 0.05 mm to about 0.5 mm. Further, apertures in the channels can be distributed within the channels with from about 1 to about 20 apertures per square centimeter with a hole spacing, measured by the shortest distance between the closest edges of adjacent apertures, from about 1 mm to about 50 mm. Also, the apertures in the channels may be arranged in a grid pattern or an offset pattern.

With reference to FIG. 8, a third embodiment of the absorbent article, specifically the acquisition distribution layer 10 of the third embodiment is illustrated. The third embodiment is similar to the second embodiment, except the apertures 14 in the non-channel portions of the acquisition distribution layer are conical with a larger surface area on the surface facing the absorbent core. These so called reverse conical apertures can make it more difficult for the fluid to enter the absorbent core via the apertures, and thus additional fluid reaches the channels.

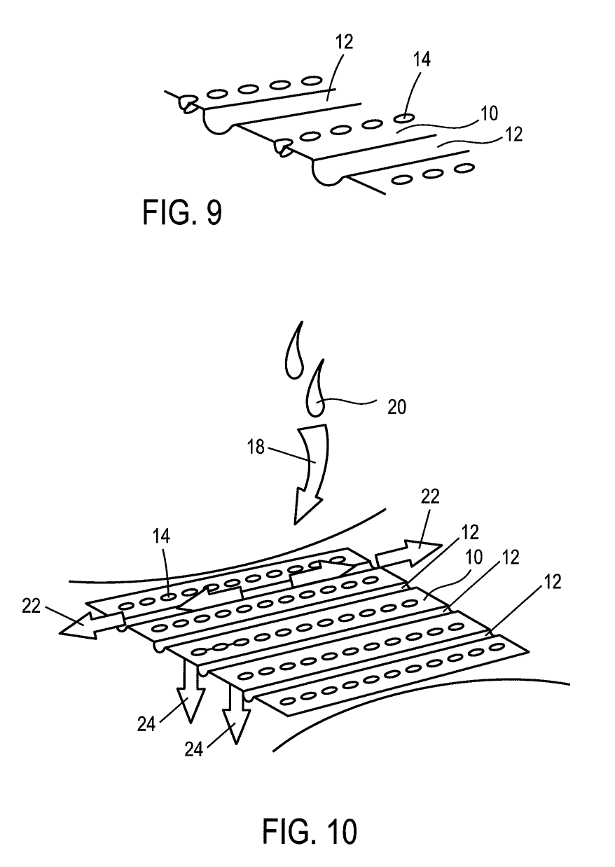

FIG. 9 shows a close up view of one longitudinal end of an embodiment of the absorbent article similar to the second embodiment of FIG. 6. The close up view provides a better illustration of the conical apertures having a larger surface area on the surface facing the top sheet. Also, the close up view provides a better illustration of the channels having a larger depth than the apertures.

FIG. 10 illustrates how fluid 20 may flow to the absorbent core through an acquisition distribution layer 10 in accordance with an embodiment of the invention. In particular, the fluid contacts the surface of the acquisition distribution layer facing the top sheet as indicated by arrow 18. As the fluid contacts the surface of the acquisition distribution layer, some of the fluid flows through the apertures 14 to contact the absorbent core as indicated by arrows 24. Simultaneously, other fluid flows longitudinally in either direction along the channels 12 to the longitudinal ends of the acquisition distribution layer as indicated by arrows 22. By some of the fluid flowing to the ends of the acquisition distribution layer via the channels, the fluid is distributed across a larger surface area of the absorbent core.

By distributing the fluid across a larger surface area of the absorbent core before saturation of the center portion of the absorbent core through the use of channels, the user is not subjected to the sensation of fluid running across a saturated central portion in pursuit of a dry portion of the absorbent core to fill. Additionally, for similar reasons, the longitudinal channels help prevent transverse liquid leakage caused by a central portion of the absorbent core becoming saturated and fluid leaking away from the saturated area in the transverse direction.

The acquisition distribution layer in accordance with the above embodiments may be made by calendaring the non-woven or polymeric film to form channels, followed by a step of punching to form apertures. For example, a non-woven or polymeric film first passes through calendar rolls that include protrusions to press longitudinal or machine direction channels into the film. Following the calendar rolls, the film passes under or over a hole punching roller. Where the film passes under a hole punching roller, downward facing holes are formed, and where the film passes over a hole punching roller, upward facing holes are formed. Any combination of calendaring or other known methods for pressing and aperturing films may be used to form different embodiments of the acquisition distribution layer. Other layers of the absorbent articles may be formed according to known methods, and the absorbent articles may be assembled according to standard methods.

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

1. An absorbent article, comprising:
a backsheet;
a topsheet;
an absorbent core between the backsheet and topsheet; and
an acquisition distribution layer comprising a first plane between the absorbent core and topsheet;
wherein the acquisition distribution layer has apertures and channels open toward the topsheet, the bottom of the channels are situated in a lower second plane, that is closer to the core than the first plane, and the channels extend in the longitudinal direction of the article, and
wherein the channels have lower permeability through the layer than other portions of the acquisition distribution layer.

2. The absorbent article according to claim 1, wherein the acquisition distribution layer includes three or more channels extending in the longitudinal direction.

3. The absorbent article according to claim 1, wherein the channels are straight, curved, jagged, or S-curved.

4. The absorbent article according to claim 1, wherein the channels have a length from about 60% to about 100% of a total length of the acquisition distribution layer.

5. The absorbent article according to claim 1, wherein the acquisition distribution layer has a length from about 50% to about 80% of a total length of the absorbent core.

6. The absorbent article according to claim 1, wherein the channels have a width of from about 1 mm to about 3 mm.

7. The absorbent article according to claim 1, wherein the channels have a depth of from 0.5 mm to about 1.5 mm.

8. The absorbent article according to claim 1, wherein the apertures situated in the first plane have a hole size from about 0.25 mm to about 1.5 mm.

9. The absorbent article according to claim 1, wherein the acquisition distribution layer has from 5 to 150 apertures per square centimeter.

10. The absorbent article according to claim 1, wherein the apertures are three dimensional and have a depth from about 0.2 mm to about 1 mm.

11. The absorbent article according to claim 1, wherein the channels contain a smaller surface area of apertures than the other portions of the acquisition distribution layer.

12. The absorbent article according to claim 11, wherein the apertures within the channels have a hole size from about 0.05 mm to about 0.5 mm.

13. The absorbent article according to claim 1, wherein the channels contain no apertures.

14. The absorbent article according to claim 1, wherein the acquisition distribution layer is formed from an apertured polymeric film.

15. The absorbent article according to claim 1, wherein the acquisition distribution layer is formed of a non-woven material.

16. The absorbent article according to claim 15, wherein the non-woven material is spunbond-meltblown-spunbond (SMS) non-woven.

17. The absorbent article according to claim 15, wherein the non-woven material has a pre-apertured hydrohead value from about 15 to about 23 mbar.

18. The absorbent article according to claim 15, wherein the channels are formed by melting the non-woven material to lower the permeability of the non-woven material in the channels.

19. The absorbent article according to claim 15, wherein the channels are substantially impermeable.

20. The absorbent article according to claim 1, wherein the absorbent article is an incontinence pad or a sanitary napkin.

21. An absorbent article, comprising:
a backsheet;
a topsheet;
an absorbent core between the backsheet and topsheet; and
an acquisition distribution layer comprising a first plane between the absorbent core and topsheet;
wherein the acquisition distribution layer has apertures and channels open toward the topsheet, the bottom of the channels are situated in a lower second plane, that is closer to the core than the first plane, and the channels extend in the longitudinal direction of the article, and
wherein the channels are impermeable and contain no apertures.
